(19) United States
(12) Patent Application Publication
(10) Pub. No.: US 2025/0058050 A1
(43) Pub. Date: Feb. 20, 2025

(54) MULTIPLE DELIVERY FLUSH SYRINGE

(71) Applicant: Becton, Dickinson and Company, Franklin Lakes, NJ (US)

(72) Inventors: Praveen Nalawade, Belgaum (IN); Syed Fareed Ahmed, Bangalore (IN); Shishir Prasad, Ramsey, NJ (US); Tirtharup Bhattacharjee, Bengaluru (IN)

(73) Assignee: Becton, Dickinson and Company, Franklin Lakes, NJ (US)

(21) Appl. No.: 18/235,109

(22) Filed: Aug. 17, 2023

(51) Int. Cl. A61M 5/31; A61M 5/14

(52) U.S. Cl. CPC A61M 5/3135; A61M 5/14; A61M 2005/1403; A61M 2005/3104; A61M 2209/06

(57) ABSTRACT

A flush syringe with a syringe coupler facilitates twice dispensing of intravenous flushing solution to a patient's vascular access device (VAD), with reduced risk of VAD infection. The syringe coupler incorporates a female, first Luer connector on its proximal end that is removably coupled to the male, barrel Luer connector of the syringe, isolating the latter connector from external contamination. The syringe coupler also has a single-use, male, second Luer connector on its distal end of the housing that is adapted for coupled insertion into the patient's VAD, for delivery of at least a portion of the flushing solution retained within the syringe. After the first delivery, the syringe coupler is de-coupled from both the VAD and the syringe. Thereafter, the syringe's unused, sterile, male, barrel Luer connector is directly coupled to the VAD for dispensing the second portion of the flushing solution.

### TECHNICAL FIELD

The present disclosure generally relates to syringes for flushing a vascular access device (VAD) of a patent, such as a patient access port. More particularly, the disclosure relates to a flush syringe that is capable of dispensing flushing solution to a patient VAD in two separate flushing procedures (e.g., before and after drug administration), each using a new, sterile port connector, reducing potential risk of VAD infection.

### BACKGROUND

Vascular access devices (VAD's) are commonly used therapeutic devices and include intravenous (IV) catheters. There are two general classifications of VAD's, peripheral catheters and central venous catheters. Several types of access hubs, ports or valves are coupled to a VAD when delivering a fluid or pharmaceutical. Some IV patient ports are activated by the frusto-conically shaped tip (e.g., a Luer connector) of a syringe barrel to allow fluid communication between the interior of the syringe and the catheter. A Luer connector is a common way to couple or join syringes, catheters, hubbed needles, IV tubes, etc. to each other. When VADs are used for medical patient care, there are potential risks of occlusions and contamination of components.

If not properly maintained, VADs can become occluded. To ensure VADs are used properly and do not become occluded, standards of practice have been developed. These standards include a cleaning procedure, which is commonly referred to as a flush procedure or flushing a catheter. VAD standards of practice usually recommend flush procedures be performed after catheter placement, before fluid infusion, and before and after drug administration, blood sampling, transfusions, and parenteral nutrition. The goal of these flush procedures is to confirm catheter patency, avoid drug incompatibilities, ensure the complete drug dose administration, prevent thrombus formation, and minimize the risk of blood stream infections. Flush procedures require diverse types and amounts of flush solutions. Commonly used flush solutions are saline and/or heparin lock solution. The type of flush solution and amount vary depending on the specific type of catheter. Flush solution volumes between 5 and 10 ml are most common but can range from 1 ml to 20 ml.

Flushing is also performed after medication administration, as residual medicine may remain in the luer portion of the drug delivery syringe and in the catheter. Without a subsequent flushing procedure, the full medication dosage may not be delivered to the patient. Some medications are administration time sensitive and should not remain in the catheter until a subsequent medicine administration flushes the residual quantity of the prior medication through the line. Therefore, sequential flushing with a second flush syringe is performed to resolve the residual medication delivery issue. Medication delivery and companion flushing through VAD delivery systems also inherently expose the delivery system to potential contamination.

Bacteria and other microorganisms may gain entry into a patient's vascular system from diverse types of patient connectors, including by way of non-limiting example, access hubs, ports or valves, upon their connection to the VAD when delivering a fluid or pharmaceutical. Each patient connector, whether an access hub, port, valve or other type of connection, is associated with some risk of transmitting a catheter related bloodstream infection (CRBSI), which can be costly and potentially lethal. In order to decrease CRBSI cases and to ensure VAD's are used and maintained correctly, standards of practice have been developed, which include disinfecting and cleaning procedures. Disinfection caps have been added to the Society for Healthcare Epidemiology of America (SHEA) guidelines and caps are also incorporated into the Infusion Nurses Standards (INS) guidelines.

In developed markets, when utilizing an IV catheter, a needleless patient connector will typically be used to close off the system and then subsequently be accessed to administer medication or other necessary fluids via the catheter to the patient. INS Standards of Practice recommend the use of a needleless connector and state that it should be “consistently and thoroughly disinfected using alcohol, tincture of iodine or chlorhexidine gluconate/alcohol combination prior to each access.” The disinfection of the needleless connector is ultimately intended to aid in the reduction of bacteria that could be living on the surface and possibly lead to a variety of catheter related complications including CRBSI. Nurses will typically utilize a 70% isopropyl alcohol (IPA) pad to complete this disinfection task by doing what is known as “scrubbing the hub.” Currently many nursing units mandate the practice of scrubbing the patient's IV connector hub, even if the connector is presently coupled to an existing disinfection cap.

Typically, four scrubbing procedures (often performed with disinfection caps) are required to administer a single drug through a VAD system in a Saline-Administration of drug-Saline (SAS) workflow process. The first disinfection scrub prepares the patient's VAD connector for drug delivery and receipt of a first flushing syringe to confirm patency within the VAD delivery system and its IV catheter. The first flushing syringe is removed after completion of the pre-flush. Thereafter a second disinfection scrub prepares the catheter hub for connection to a medication delivery syringe. A third disinfection scrub prepares the catheter hub for connection of a second flushing syringe to complete a post-medication delivery flush. Lastly, after removal of the second flushing syringe, a fourth disinfection scrub is performed, followed by sealing of the catheter hub with a cap or a locking syringe. In the case of locking the IV catheter with fluid other than Saline (e.g. heparin) or Saline-Administration of drug-Saline-Heparin (SASH) workflow process, a third, heparin flushing procedure is required after the second saline flush, which requires a third flush syringe and a fifth disinfection scrub. When more than one drug is administered to a patient, during the workflow process, each additional drug dose is followed by hub disinfection before initiating the next dispensing task.

Therefore, throughout the sequence of procedures necessary to prevent occlusions within VAD systems and to administer medication to a patient there are contamination and microorganism transmission risks that can cause a CRBSI every time a syringe is connected or disconnected from the patient's VAD connector. Best IV catheter contamination practices prohibit re-use of a syringe connector to a patient port connector.

Returning now to procedures for preparing a patient port for drug administration, after the initial IV port scrubbing, followed by flushing with a first, sterile, single-use flush syringe, the practitioner again scrubs the IV port and administers a dosage of medication fluid with a new, sterile, single-use drug syringe, followed by a post-administration scrubbing and flushing with a second, sterile, single-use flush syringe. The initial IV port flush, administration of intravenous medication, and the second IV flush following medication administration typically utilizes three separate, single-use syringes in clinical practice. There is risk that practitioners might re-use a contaminated, single use flushing syringe to perform the pre- and the post-medication delivery flushing during medication administration. Re-use of a flush syringe that was previously connected to a patient IV port increases the risk of transmitting a catheter related bloodstream infection (CRBSI) to the patient.

However, it is appreciated that reducing the number of syringes necessary to flush and administer drugs through a patient's IV port, while conforming to CRBSI reduction practices is beneficial in many ways. Syringe use reduction reduces clinician time and effort necessary to open a fewer number of sterile syringe packages; it also reduces syringe stocking inventory and associated purchase and medical waste disposal costs.

### SUMMARY

A flush syringe incorporates a syringe coupler that facilitates dispensing of intravenous flushing solution to a vascular access device (VAD) of a patient two times (e.g., before and after drug administration), with reduced risk of VAD infection. The syringe embodiments of the present disclosure connect an unused, sterile Luer connector for each of the two flushing procedures. The distal axial end of the syringe coupler provides the first, sterilized Luer connector for coupling to a corresponding hub Luer connector of the VAD and the distal end of the syringe coupler establishes a sterile, fluid seal with the corresponding barrel Luer connector of the syringe barrel.

When the syringe coupler is removed/de-coupled from the syringe's still sterile, barrel Luer connector, the latter is directly coupled to a now re-disinfected hub Luer connector of the VAD. More specifically, the syringe coupler has a female, first Luer connector on its proximal end that is removably coupled to the male, barrel Luer connector of the syringe, which isolates the latter and flushing solution within the syringe barrel from environmental contamination. A male, second Luer connector on its distal end of the housing that is adapted for coupled insertion into the patient's VAD, for delivery of at least a portion of the flushing solution retained within the syringe. Typically, after the first VAD flushing procedure, a clinician disinfects the VAD and administers one or more drugs to the patient through the VAD, disinfecting the VAD after each new drug administration. After the first flushing procedure, the syringe coupler is de-coupled from both the VAD and the male, barrel Luer connector of the syringe. Thereafter, the unused, sterile barrel Luer connector is coupled directly to the VAD for dispensing the second portion of the flushing solution. Each VAD connection by the syringe is done with a fresh, sterile connector. No VAD connector of the syringe is used twice. Thus, a single IV flush syringe can be used to perform both the pre- and post-medication flushing of a patient's IV port. This can lead to a fifty-percent reduction in the number of flush syringes needed to administer drugs to a patient, while still adhering to best CRBSI reduction practices. The syringe use reduction also reduces associated syringe inventory and medical waste disposal costs. Some flush syringe embodiments incorporate tamper-evident end caps that visually identify whether the first Luer connector was previously used. Other flush syringe embodiments incorporate a twist-lock mechanism that passively separates the syringe coupler from the syringe barrel after initial use of the first Luer connector and its removal from the VAD.

One aspect of the present disclosure pertains to flush syringes for intravenous fluid administration to a patient. In some embodiments, the syringe is pre-filled with flushing solution and packed in sterile packaging. The syringe includes a syringe barrel with a distal end, an open proximal end, and a syringe cavity within the barrel. A syringe plunger is translatable within the syringe cavity. A male, barrel Luer connector is oriented on the distal end of the syringe barrel; it has a male, barrel Luer tip defining an outlet lumen in communication with the syringe cavity. A syringe coupler, coupled to the syringe, includes a housing having proximal and distal axial ends, and a central axis, with a female, first Luer connector on the proximal end of the housing, coaxial with the central axis; the first Luer connector has a first lumen defined therein. The syringe coupler also has a male, second Luer connector on the distal end of the housing that is coaxial with the central axis; it has a male Luer tip with a second lumen defined therein. The first and second lumens are in mutual fluid communication. A storage cap is coupled to the housing, covering the male, second Luer connector and sealing the second lumen. The female, first Luer connector of the housing is adapted for coupled receipt to male, barrel Luer connector, for delivery of flushing solution retained within the syringe cavity through the outlet lumen of the male, barrel Luer connector, into the first and second lumen lumens of the housing. The male, second Luer connector of the housing is adapted for coupled insertion into a corresponding needle-free IV connector of a patient or other type of VAD, for delivery of at least a portion of the flushing solution retained within the syringe cavity into the IV connector. The syringe coupler is selectively removeable from the male, barrel Luer connector, thereby allowing direct coupling of the latter to the corresponding needle-free IV connector for delivery of additional flushing solution retained within the syringe cavity into the IV connector.

Another aspect of the disclosure pertains to a method for manufacturing the aforementioned flush syringe. During such syringe manufacture, a syringe barrel is provided with a male, barrel Luer connector oriented on a distal end of the syringe barrel, an open proximal end, and a syringe cavity within the barrel. The syringe's barrel Luer connector has a male, barrel Luer tip defining an outlet lumen in communication with the syringe cavity. A syringe coupler is provided, which includes housing having proximal and distal axial ends and a central axis. A female, first Luer connector is oriented on the proximal end of the housing, coaxial with the central axis and has a first lumen defined therein. A male, second Luer connector is oriented on the distal end of the housing, coaxial with the central axis, and has a male Luer tip with a second lumen defined therein. The first and second lumens are in fluid communication with each other. The female, first Luer connector is coupled to the barrel Luer connector. In some embodiments, a selectively removeable storage cap is coupled to the housing, covering the male, second Luer connector of the syringe coupler and sealing the second lumen. In some embodiments, during manufacture of the syringe, the syringe cavity is prefilled with a flushing solution. Thereafter, a translatable syringe plunger is inserted into the syringe barrel within the syringe cavity. In some embodiments, after insertion of the syringe plunger, the assembled flush syringe is sterilized and packaged in a sterile packing container. In some embodiments, plural flush syringes are packaged in a shared, sterile packing container.

Yet other aspects of this disclosure pertain to a method flushing a patient port during intravenous fluid administration to a patient by using the aforementioned and described flush syringe. When performing this flushing method, it is contemplated that the administering clinician will adhere to approved medical protocols for reducing likelihood of VAD contamination. The clinician disinfects the hub of a patient port having a hub Luer connector or other VAD. After removal of the flush syringe from its sterile packaging and subsequent removal of the storage cap from the syringe coupler, the male, second Luer connector of the syringe coupler is coupled to the hub Luer connector of the patient port. By depressing the syringe plunger, a first portion of pre-filled flushing solution retained within the syringe cavity is dispensed into the patient port, through the male, second Luer connector of the syringe coupler. The male, second Luer connector is de-coupled from the hub Luer connector and the hub of the patient port is disinfected. Thereafter, one or more drugs are administered through the hub with drug dosing syringes, disinfecting the hub after delivery of each drug. The syringe coupler is de-coupled from the syringe by decoupling the female, first Luer connector from the male, barrel Luer connector of the syringe. Thereafter, the syringe coupler is discarded. In order to prepare for the second VAD flushing procedure, the hub of the patient port is disinfected and the male, barrel Luer connector is coupled to the hub Luer connector of the patient port and a second portion of the pre-filled flushing solution retained with the syringe cavity is dispensed through the male, barrel Luer connector into the patient port by depressing the syringe plunger. The male, barrel Luer connector is thereafter de-coupled from the hub Luer connector of the patient port, and the syringe is discarded. The hub of the patient port is disinfected again and capped with a sterilized hub cap.

### BRIEF DESCRIPTION OF THE DRAWINGS

Exemplary embodiments of the disclosure are further described in the following detailed description in conjunction with the accompanying drawings, in which:

FIG. 1 is an isometric view of an embodiment of a flush syringe of the present disclosure;

FIG. 2 is a partial axial cross section of the syringe of FIG. 1;

FIG. 3 is an isometric view of the syringe of FIG. 1 coupled to an exemplary needle free connector (NFC) patient port type of VAD, for a first delivery of flushing solution to the VAD;

FIG. 4 is an enlarged, axial cross-sectional view of the coupled syringe coupler/syringe and the patient port of FIG. 3;

FIG. 5 is an isometric view of the syringe of FIG. 3 after the flushing the patient port with a portion of the flushing solution previously contained within the syringe, and decoupling of the syringe coupler from both the patient port and the syringe barrel;

FIG. 6 is an isometric view of the syringe of FIG. 3, after coupling its barrel Luer connector directly to the patient port, for flushing the patient port a second time with residual flushing solution remaining in the syringe after the first flushing procedure;

FIG. 7 is a partial isometric elevational view of another embodiment of a flush syringe of the present disclosure, having a tamper-evident peel cap;

FIG. 8 is a partial cross-sectional view of the flush syringe of FIG. 7;

FIG. 9 is an isometric view of another embodiment of a flush syringe, prior to removal of its tamper-evident storage cap and coupling to a patient port;

FIG. 10 is a partial exploded elevational view of the flush syringe of FIG. 9;

FIG. 11 is a partial isometric view of the distal end of the flush syringe of FIG. 9;

FIG. 12 is a partial, axial cross-sectional view of the flush syringe of FIG. 9, showing a first end cap embodiment for sealing the second lumen of the syringe coupler;

FIG. 13 is a partial, axial cross-sectional view of the flush syringe of FIG. 9, showing a second end cap embodiment for sealing the second lumen of the syringe coupler;

FIG. 14 is a partial, isometric view of the flush syringe of FIG. 9 after removal of a tamper-evident, frangible, tear-off ring;

FIG. 15 is a partial, isometric view of the flush syringe of FIG. 9, after removal of the tamper-evident, frangible, tear-off ring and attachment of its syringe coupler to a patient port;

FIG. 16 is a partial, isometric view of the flush syringe of FIG. 9, after removal of the syringe coupler from the syringe;

FIG. 17 is an isometric view of the syringe of FIG. 9, after coupling its barrel Luer connector directly to the patient port, for flushing the patient port a second time with residual flushing solution remaining in the syringe after the first flushing procedure;

FIG. 18 an isometric view of another embodiment of a flush syringe of the present disclosure, having a twist-lock mechanism for passive separation of its syringe coupler from its syringe barrel;

FIG. 19 is a partial, isometric view of the distal end of the flush syringe of FIG. 18;

FIG. 20 is a partial, axial cross-sectional view of the flush syringe of FIG. 18;

FIG. 21 is an enlarged, layered view of the distal end of the flush syringe of FIG. 18, prior to removal of its storage cap and attachment of its syringe coupler to a patient VAD; and

FIG. 22 is an enlarged, layered view of the distal end of the flush syringe of FIG. 18, during detachment of its syringe coupler from the barrel Luer connector of the syringe.

To facilitate understanding, identical reference numerals have been used, where possible, to designate identical elements that are common to the figures. The figures are not drawn to scale.

### DETAILED DESCRIPTION

Aspects of embodiments of the flush syringes disclosed herein facilitate two separate, discharges and deliveries of intravenous flushing solution contained within a flush syringe to a vascular access device (VAD) of a patient, with reduced risk of VAD contamination. Each of the two separate syringe connections to the VAD is done with a new, sterile VAD connector. Two separate discharges of uncontaminated flushing solution into a patient's VAD, before and after medication dosing, is possible because the flush syringes disclosed herein have two separate, sterilized Luer connectors available for coupling to the VAD's hub connector. In this way, a clinician has the ability first to flush a patient's VAD through a male, Luer connector on a distal end of a syringe coupler that is oriented between and in fluid communication with both the VAD and the flushing solution contained within the flush syringe. The syringe coupler has a female, Luer connector on its proximal end that is removably coupled to the male, barrel Luer connector of the syringe, which isolates the latter and flushing solution within the syringe barrel from environmental contamination. After the initial flushing procedure, the male, Luer connector of the syringe coupler is de-coupled from the patient's VAD. Typically, after the first VAD flushing procedure, a clinician disinfects the VAD and administers one or more drugs to the patient through the VAD, disinfecting the VAD after each new drug administration. After completion of drug administration, the female, Luer connector of the syringe coupler is de-coupled from the uncontaminated, male, barrel Luer connector of the syringe. Thereafter, the uncontaminated barrel Luer connector is coupled directly to the VAD for dispensing the second portion of the flushing solution. Some embodiments disclosed herein incorporate tamper-evident caps to provide a visual indicator to a clinician whether the syringe coupler has been previously used. Other embodiments incorporate a twist-lock mechanism that passively separates the syringe coupler from the syringe barrel when the male, second Luer connector is disconnected from a patient port, so that the syringe coupler cannot be reused. Yet other embodiments incorporate both a tamper-evident cap and a twist-lock mechanism.

In this disclosure, a convention is followed wherein the distal end of the device is the end closest to a patient, e.g., for delivery of one or more drugs to the patient, and the proximal end of the device is the end away from the patient and closest to a clinician or other medical practitioner. With respect to terms used in this disclosure, the following definitions are provided.

As used herein, the use of “a,” “an,” and “the” includes the singular and plural.

As used herein, the term “Luer connector” refers to a connection collar that is the standard way of attaching syringes, catheters, hubbed needles, IV tubes, etc. to each other. The Luer connector consists of male and female interlocking tubes, slightly tapered to hold together better with even just a simple pressure/twist fit. Luer connectors can optionally include an additional outer rim of threading, allowing them to be more secure. The Luer connector male end is associated with a flush syringe and can interlock and connect to the female end located on the vascular access device (VAD). A Luer connector also has a distal end channel that releasably attaches the Luer connector to the hub of a VAD, and a proximal end channel that releasably attaches the Luer connector to the barrel of a syringe.

As used herein, ISO 80369-7:2016 defines a specification for standard Luer connectors including a 6% taper between the distal end and the proximal end. A male standard Luer connector increases from the open distal end to the proximal end. A female standard Luer connector decreases from the open proximal end to the distal end. According to ISO 80369-7:2016, a male standard Luer connector has an outer cross-sectional diameter measured 0.75 mm from the distal end of the tip of between 3.970 mm and 4.072 mm. The length of the male standard Luer taper is between 7.500 mm to 10.500 mm. The outer cross-sectional diameter measured 7.500 mm from the distal end of the tip is between 4.376 mm and 4.476 mm. As used herein, the phrases “male standard Luer connector” and “female standard Luer connector” shall refer to connectors having the dimensions described in ISO 80369-7, which is hereby incorporated by reference in its entirety.

As would be readily appreciated by skilled artisans in the relevant art, while descriptive terms such as “tip”, “hub”, “thread”, “protrusion/insert”, “tab”, “slope”, “wall”, “top”, “side”, “bottom” and others are used throughout this specification to facilitate understanding, it is not intended to limit any components that can be used in combinations or individually or to require specific spatial orientations, to implement various aspects of the embodiments of the present disclosure.

Before describing several exemplary embodiments of the disclosure, it is to be understood that the disclosure is not limited to the details of construction or process steps set forth in the following description. The disclosure is capable of other embodiments and of being practiced or being conducted in many ways.

The matters exemplified in this description are provided to assist in a comprehensive understanding of exemplary embodiments of the disclosure. Accordingly, those of ordinary skill in the art will recognize that various changes and modifications of the embodiments described herein can be made without departing from the scope and spirit of the disclosure. Also, descriptions of well-known functions and constructions are omitted for clarity and conciseness.

In an exemplary implementation of the embodiments of present disclosure, a barrel of a syringe includes a distal end having a needleless connection. In one or more embodiments, the needleless connection includes at least one thread and other features in all combinations allowing it to interface with a corresponding thread or plurality of threads of a corresponding connector.

According to further exemplary implementations of the embodiments of the present disclosure, configuration of structural elements making up the needleless connector include a collar protruding from the distal end of the barrel, the collar comprising at least one thread to connect to the corresponding thread or plurality of threads of a corresponding connector.

According to still further exemplary implementations of the embodiments of the present disclosure, the collar or the needleless connector bends or elastically deforms to allow better interference fit compliance with corresponding connectors.

According to still further exemplary implementations of the embodiments of the present disclosure, the needleless connector may comprise female threads that are sized and have a thread pattern that will engage with a standard IS0594-2 type of male fitting and/or male threads that are sized and have a thread pattern that will engage with a standard IS0594-2 type of female fitting. An example of an IS0594-2 type of fitting is a Q-style fitting.

In one or more embodiments, a female connector may be selected from the group consisting essentially of: needle-type connectors (for direct injection into a patient or insertion into a drug vial for aspiration of a drug dose therefrom), needle-free connectors, catheter Luer connectors, stopcocks, and hemodialysis connectors. In one or more embodiments, the needleless connector is selected from a Q-Syte connector, MaxPlus, MaxPlus Clear, MaxZero, UltraSite, Caresite, InVision-Plus, Safeline, OneLink, V-Link, ClearLink, NeutraClear, Clave, MicroClave, MicroClave Clear, Neutron, NanoClave, Kendall, Nexus, InVision, Vadsite, Bionector, etc.

In one or more embodiments, the male connector may be an intravenous tubing end or a stopcock.

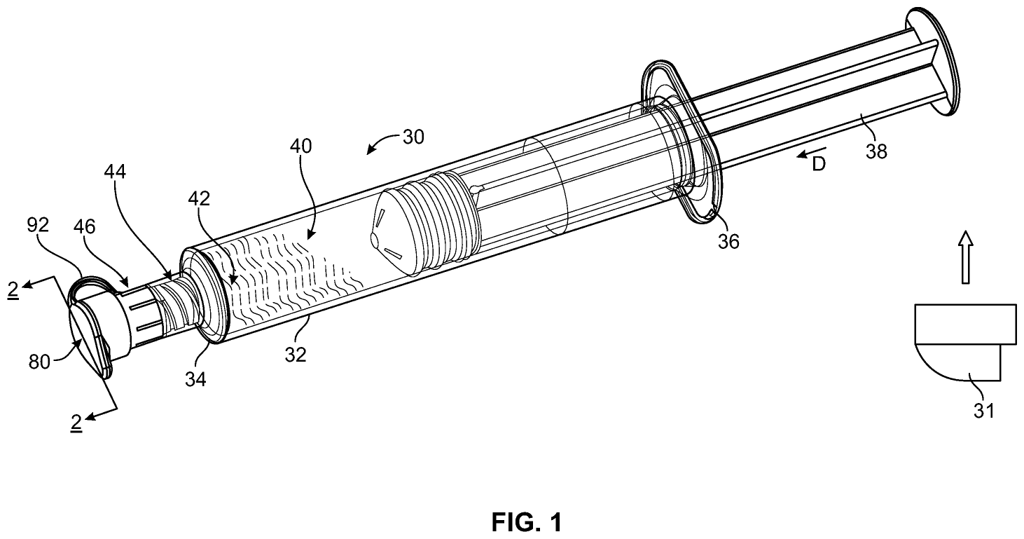

Referring now to the drawings, the first aspect of the present disclosure is shown in FIGS. 1-6, wherein a flush syringe 30, now removed from its sterile packaging 31, comprises a syringe barrel 32, which defines a distal end 34 an open, proximal end 36, and a syringe plunger 38 that is retained within an internal syringe cavity 40. In some embodiments, the syringe cavity 40 is pre-filled with a flushing solution 42 during syringe manufacture. In some embodiments, the volume of flushing solution 42 that is retained within the syringe cavity 40 is 10 ml. The conical profile, distal end 34 of the syringe barrel 32 incorporates a threaded, male, barrel Luer connector 44, which is connected to a syringe coupler 46. The barrel Luer connector 44 includes a male, barrel Luer tip 50, with a tapered, outer circumferential surface 51 terminating at a distal end surface 52. An outlet lumen 54, formed in the distal end surface 52 of the barrel Luer tip 50 is in fluid communication with the syringe cavity 28. The threaded, male, barrel Luer connector 44 has a collar 48 with at least one internally facing thread 56 that circumscribes the barrel Luer tip 50. The syringe plunger 38 advances in the direction of the arrow D, to dispense flushing solution 42 under pressure from the syringe cavity 40 through the outlet lumen 54 of the barrel Luer connector 44.

As noted, in some embodiments, such as the flush syringe 30 of FIG. 1, the syringe cavity 40 is pre-filled with flushing solution 42, with a proximal axial end of the syringe coupler 46 coupled to the barrel Luer connector 44, and a flip cap 80 (also referred to herein as an end cap) is coupled to a distal end of the syringe coupler to retain the flushing solution within the syringe. Typically, the assembled, pre-filled, flush syringe 30, syringe coupler 46 and flip cap 80 are prepackaged the sterile packaging 31. In some embodiments, plural flush syringes 30 are separately packaged in a shared, sterile packing container. An exemplary flush syringe is shown and described in U.S. Pat. No. 8,632,521B2, entitled “Syringe Assembly”, the entire contents of which is hereby incorporated by reference in all legal jurisdictions whose patent laws permit such incorporation.

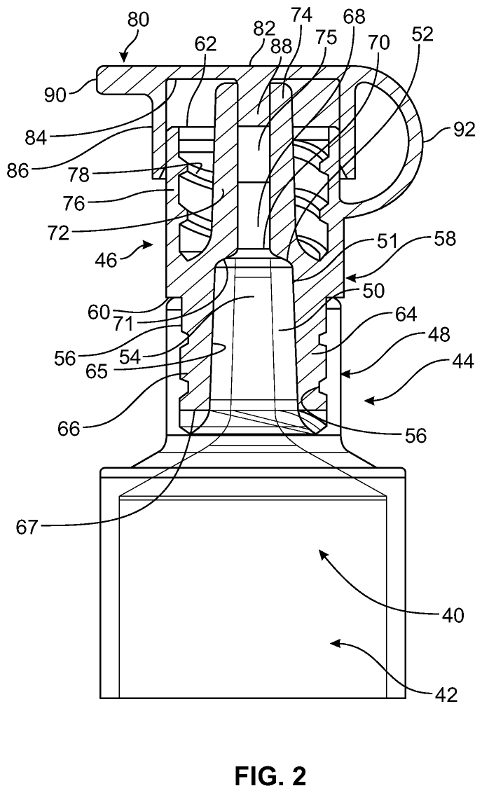

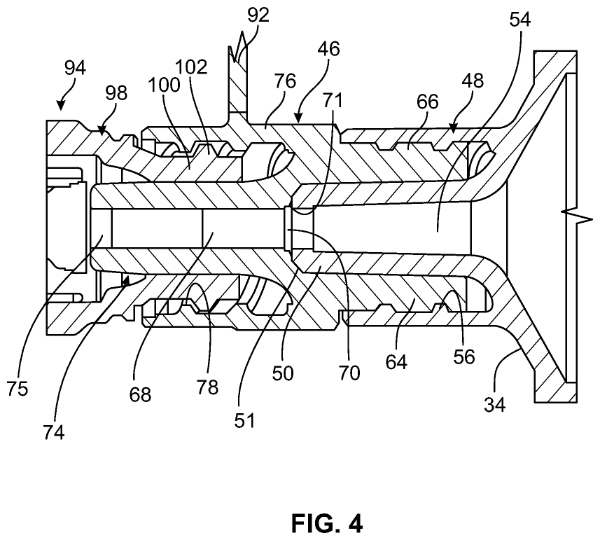

Referring more particularly to the cross-section FIGS. 2 and 4, the syringe coupler 46 comprises a housing 58, with proximal 60 and distal 62 axial ends. Housing 58 defines a central axis. A threaded, female, first Luer connector 64 on the proximal end 60 of the housing 58 is oriented coaxial with the central axis and defines a female Luer taper surface 65 and external threads 66 that terminate at a proximal end surface 67. The external threads 66 of the female, first Luer connector 64 are in mating engagement with the corresponding internally facing threads 56 formed within the collar 48 of the male, barrel Luer connector 44. A first lumen 68 of the female, first Luer connector 64 has an inlet 70 formed in a seating surface 71 of the housing 58. The first lumen 68 is in fluid communication with the outlet lumen 54 of the barrel Luer connector 44. A threaded, male, second Luer connector 72 on the distal end 62 of the housing 58 is oriented coaxial with the central axis and includes a tapered, male Luer tip 74. A second lumen 75 is formed within the male Luer tip 74 and is in fluid communication with the co-joined, first lumen 68 of the female, first luer connector 64. The male, second Luer connector 72 incorporates a collar 76 that defines at least one internally directed thread 78.

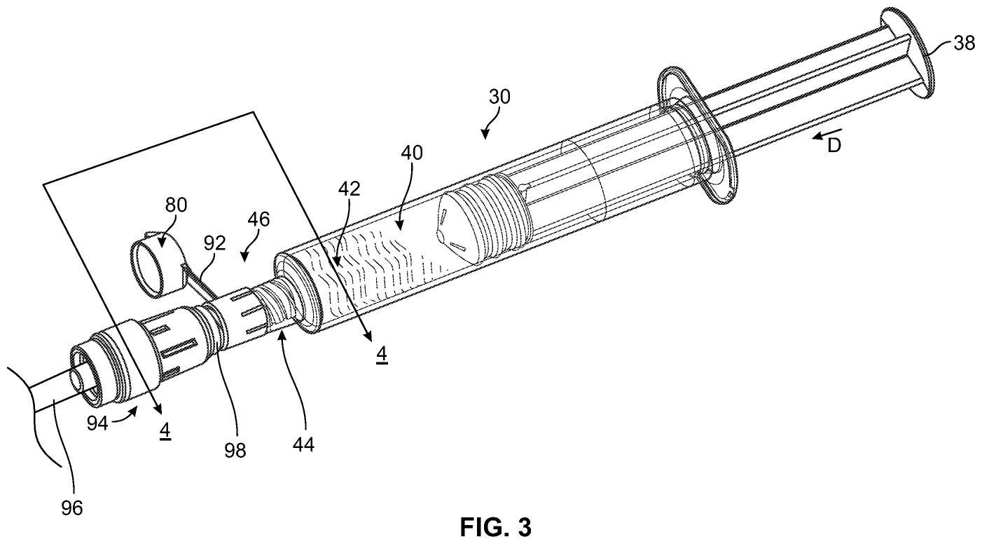

Referring now to FIGS. 1-3, storage or end cap of the syringe coupler 46 is a flip cap 80, having an outer, distal end surface 82, an inner surface 84 and a circumferential wall 86. When the flip cap 80 covers the distal end 62 of the housing 58, of the syringe coupler 46, a male sprue 88 that is formed on the inner surface 84 is inserted within the second lumen 75 of the male, second Luer connector 72, preventing leakage of flushing solution 42 out of the syringe. The closed flip cap 80 also maintains sterility of the male, second Luer connector 72. A lift tab 90 facilitates separation of the flip cap 80 from the syringe coupler 46, exposing the male, second Luer connector, as shown in FIG. 3. In some embodiments, a tamper-evident tape or shrink seal is applied to the flip cap 80 and the housing 58, giving a visual indication to clinicians whether the syringe 30 has been previously used. A cap hinge 92 is coupled to the flip cap 80 and the housing 58, for tethering the flip cap to the coupler housing. The cap hinge 92 is integrally formed with the housing 58 and the flip cap 80; in other embodiments, one or more of these components are separately fabricated and coupled together.

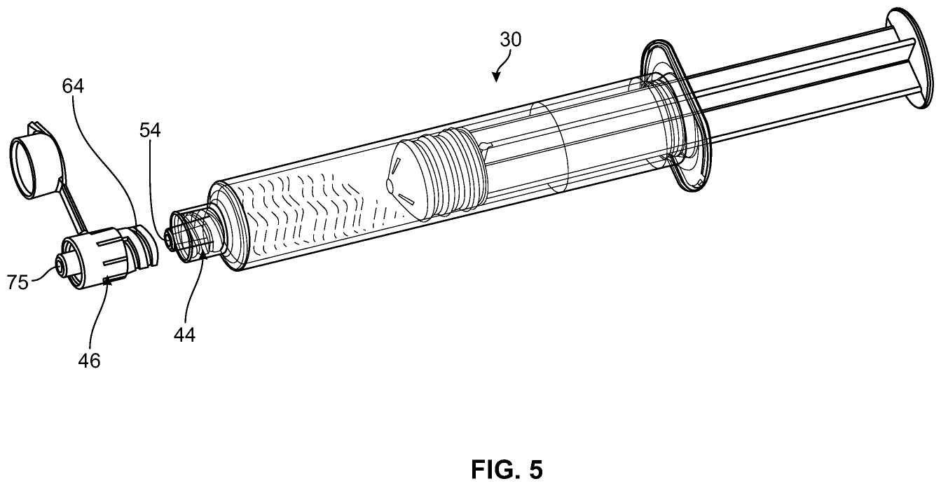

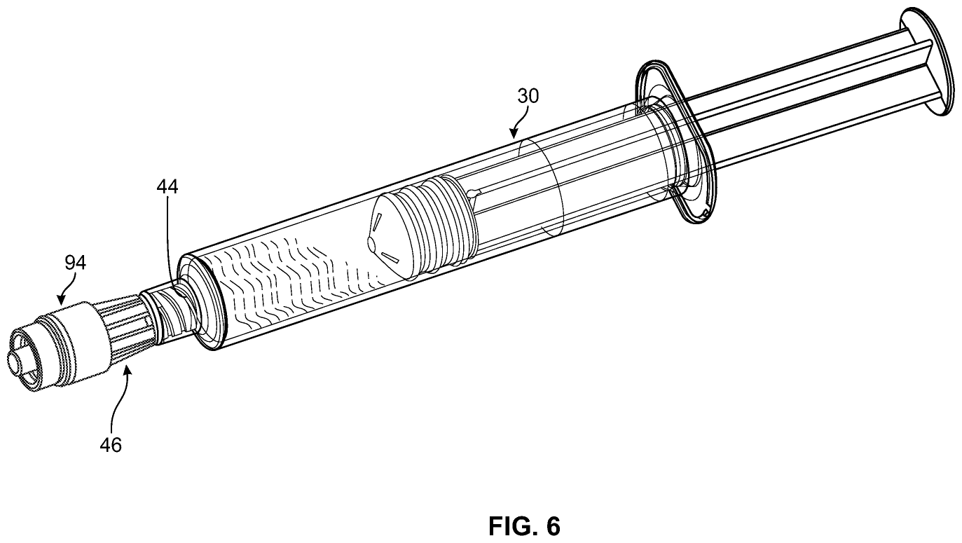

FIGS. 3 and 4 depict coupling of the male, second Luer connector 72 of the syringe coupler 46 to a patient port, such as the needle free connection (NFC)-type, patient port 94. The patient port 94 has a patient IV catheter 96, a hub 98, and a threaded, female, hub Luer connector 100 with external threads 102. At least one internally directed thread 78 formed in the collar 76 of the male, second Luer connector 72 engages the external threads 102 of the female, hub Luer connector 100. During SASH administration, after initial flushing of patient port 94, with the flush syringe 30, the syringe coupler 46 is unscrewed from patient port and in turn is unscrewed from the barrel Luer connector 44 of the of the syringe (FIG. 5). After administration of medication and disinfection of patient port 94, the barrel Luer connector 44 is affixed directly to the patient port for a follow-up flush (FIG. 6). The male, second Luer connector 72 and the male, barrel Luer connector 44 of FIGS. 2 and 4 have compatible construction; either of these male connectors intentionally is capable of coupling to a corresponding hub Luer connector of a patient port, such as the needle free connection (NFC)-type, patient port 94 of FIGS. 3, 4 and 6, for flushing the port.

The flush fluid pathway established within the syringe coupler 46 enables delivery of flushing solution to a patient port, such as the patient port 94, when the port is coupled to the second, male Luer connector 72, in the same fashion as when connected directly to the barrel Luer connector 44. In the embodiment of FIGS. 1-6, the first lumen 68 and the second lumen 75 of the syringe coupler's housing 58 are co-joined to form a commonly shared fluid pathway with a flow direction originating at the inlet 70 and seating surface 71 of the female, first Luer connector 64 of the housing 58 and discharging out of the distal end of the male Luer tip 74 of the male, second Luer connector 72. Typically, in the initial flush through the male, second Luer connector 72, 5 ml of flushing solution is dispensed from the syringe cavity 40 into the hub Luer connector 100, which is half of the syringe cavity's 10 ml capacity. After connecting the barrel Luer connector 44 to the hub Luer connector 100, the remaining flush solution in the syringe cavity 40 is dispensed into the hub Luer connector.

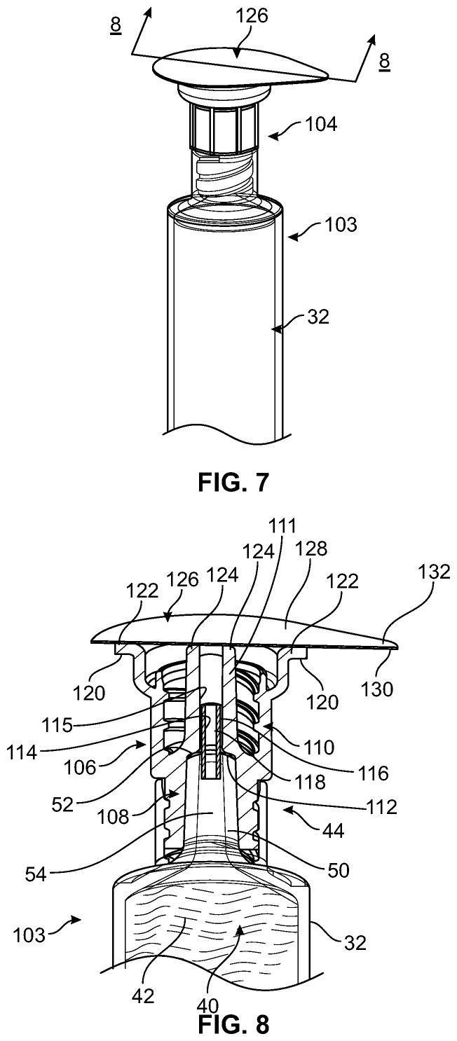

FIGS. 7 and 8 depict another embodiment of a flush syringe 103, with a syringe coupler 104 that incorporates a peel lid 126 type of storage or end cap for covering and sealing its distal end. The peel lid 126 advantageously provides a tamper-evident visual indicator to a clinician whether sterility of the syringe coupler 140 is compromised. The syringe coupler 104 has a housing 106, with a first, female Luer connector 108 and with a second, male Luer connector 110 having a male Luer tip 111. A seating surface 112 of the housing 106 defines a first lumen 114 of the first, female Luer connector 108. The male Luer tip of the second, male Luer connector 110 defines a second lumen 115. The first lumen 114 and the second lumen 115 of the syringe coupler's housing 106 are co-joined to form a commonly shared fluid pathway with a flow direction originating at the seating surface 112 of the female, first Luer connector 108 and discharging out of the distal end of the male Luer tip 111 of the male, second Luer connector 110. A hollow sleeve 116 is retained within the shared fluid pathway of the housing 106, formed by the co-joined First lumen 114 and second lumen 115. In some embodiments, the hollow sleeve 116 is bonded to the housing 106 within the shared fluid pathway. A proximal axial end of the hollow sleeve 116 projects out of the seating surface 112 and the first lumen 114 towards the proximal axial end of the housing 106. The proximal axial end of the hollow sleeve 116 is inserted within the outlet lumen 54 of the male, barrel Luer tip 50. In some embodiments, insertion of the hollow sleeve 116 into the outlet lumen 54 effectively isolates the outlet of the barrel Luer connector 44 and the syringe cavity 40 from external contamination. By insertion of the proximal axial end of the hollow sleeve 116 into the outlet lumen 54, a flow channel or pathway is created for delivery of the flushing solution 42 retained within the syringe cavity 40, through a hollow sleeve lumen 118 of the hollow sleeve, into the second lumen 115 of the housing's male, second Luer connector 110. In other embodiments of the syringe coupler 104, the construction of the respective Luer connectors 108 and 110 and their internal fluid passageway connections are identical to those of the syringe coupler 46.

The distal end of housing 106 of the flush syringe 103 incorporates a circumferential cup 120 that projects radially away from the housing's central axis and axially away from the distal end of the housing. The circumferential cup 120 defines a distal axial surface 122 that along with the distal tip surface 124 of the male, second Luer connector 110 are covered by a peel lid 126. In one or more embodiments, peel lid 126 is a peelable seal, which comprises an aluminum or multi-layer polymer film peel back top. The peel lid 126 comprises a distal outer surface 128 as well as a proximal inner surface 130 that is bonded or otherwise adhered to the distal axial surface 122 of the circumferential cup 120 and the distal tip surface 124 of the male, second Luer connector. Seal composition of the peel lid 126 provides a sufficient environmental seal between its aluminum or polymer film of its proximal, inner surface 130 and the opposing, joined distal axial surface 122 of the circumferential cup 120, along with the opposing, joined distal tip surface 124 of the male, second Luer connector 110, at a range of temperatures, pressures, and humidity levels expected within a medical treatment facility. In some embodiments, the peel lid 126 is heat-sealed or induction-sealed to the circumferential cup 120, along with the opposing, joined distal tip surface 124 of the male, second Luer connector 110. In other embodiments, the peel lid 126 is sealed to the circumferential cup 120, along with the opposing, joined distal tip surface 124 of the male, second Luer connector 110 with pressure or thermally sensitive adhesive. The adhered peel lid 126 maintains sterility of the male, second Luer connector 110 and prevents flushing solution leakage out of the second lumen 115 of that connector. Peel tab 132 facilitates removal of the peel lid 126 from the housing 106, to expose the male, second Luer connector 110. In summary, the peel lid 126 minimizes entry of potential particulate hazard and also provides a substantially impermeable enclosure for the distal end of the housing 106, provides leak prevention, and/or maintains a sealed, sterilized environment within the housing.

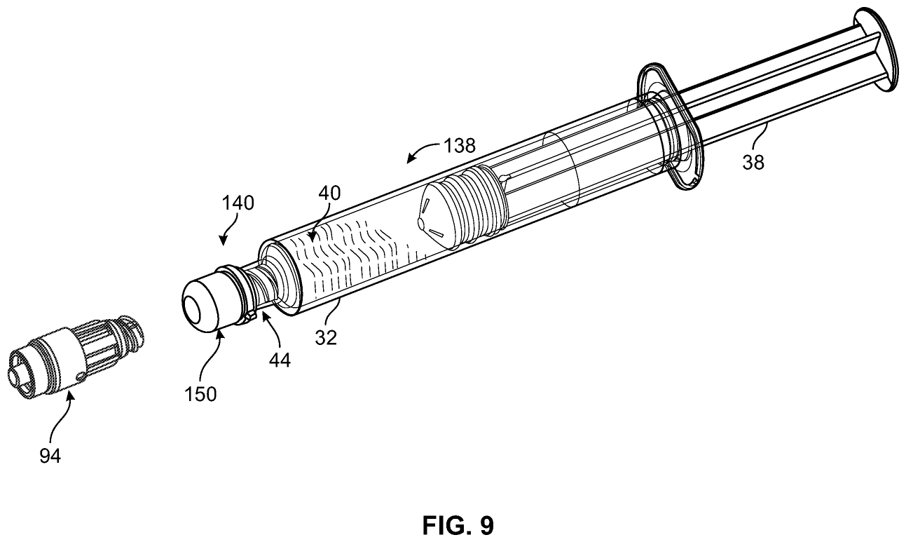

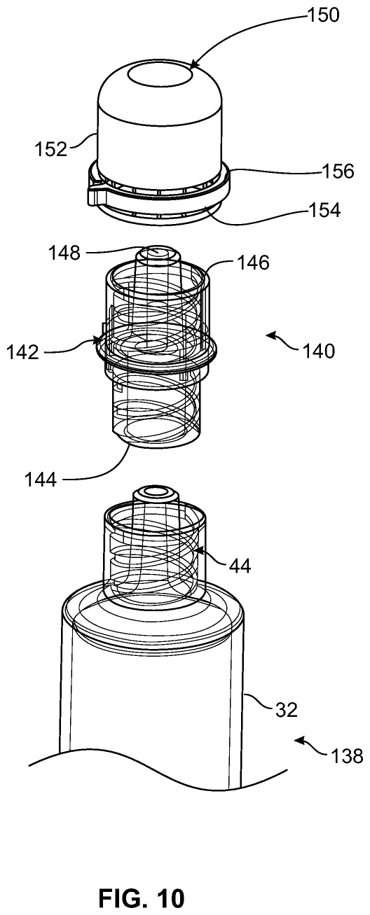

FIGS. 9-17 depict another embodiment of a flush syringe 138, with a syringe coupler 140 that incorporates a tamper-evident type of storage or end cap assembly 150 for covering and sealing its distal end. The end cap assembly 150 advantageously provides a tamper-evident visual indicator to a clinician whether sterility of the syringe coupler 140 is compromised. The syringe barrel 32, syringe plunger 37 and the barrel Luer connector 44 of the flush syringe 138 are identical to those of the previously described flush syringe embodiments 30 and 103 of FIGS. 1-8. The syringe coupler 140 comprises a housing 142, with a female, first Luer connector 144 on a proximal end of the housing that is coupled to the barrel Luer connector 44. The housing 142 further comprises a male, second Luer connector 146 with a second lumen 148 for discharging flushing solution out of the housing when connected to a patient port 94. In some embodiments, syringe coupler 140 is constructed of clear or tinted polymer that allows visual inspection of its internal structure. In various embodiments, internal structure and fluid passage through the syringe coupler 140 from the internal syringe cavity 40 into a patient port 94 is identical to those of the syringe coupler embodiments 46 and 104 of FIGS. 1-8.

In some embodiments, plural syringe couplers 140 are coupled and stacked serially, facilitating more than two flushing procedures from the same syringe barrel 32. In use, the most distal syringe coupler 140 is connected to a patient port, for the initial flushing procedure and discarded upon completion of that procedure. The next available, most distal syringe coupler 140 in the stack is used to perform the second flushing procedure, etc.

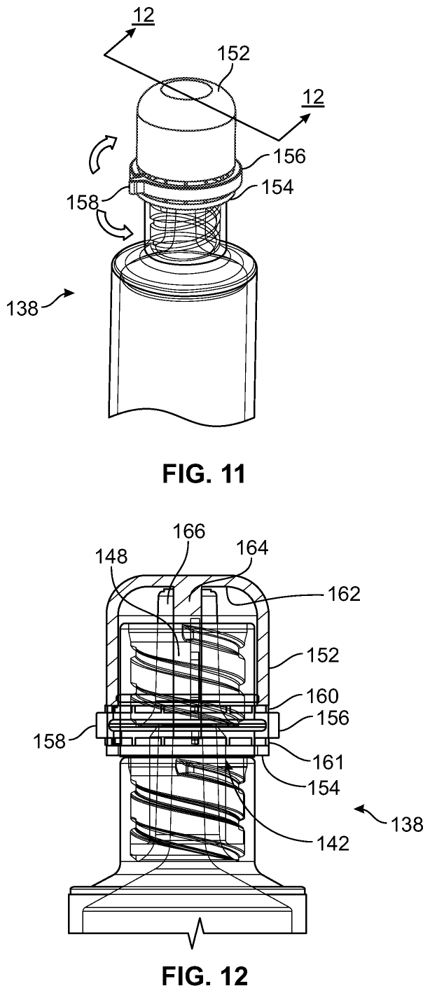

One embodiment of the tamper evident, end cap assembly 150 comprises an end cap embodiment 152 that covers the male, second Luer connector 146 and its second lumen 148, a snap-on band 154, and a split-ring type, tear-off ring 156. The snap-on band 154 snaps over and is rigidly engaged with the outer circumferential surface of the syringe coupler 140. The tear-off ring has a finger tab 158 on one end of its split ring structure. In this embodiment, the end cap assembly 150 is a monolithic polymer structure, formed by injection molding, with a plurality of integrally formed first shear-off connectors 160 joining the end cap 152 and the tear-off ring 156, and a plurality of integrally formed, second shear off connectors 161 joining the tear off ring to the snap-on band 154. As shown by the double arrows in FIG. 11, separating the finger tab end of the tear-off ring from its opposed split end breaks both pluralities of shear-off connectors 160 and 161, allowing end cap 152 removal.

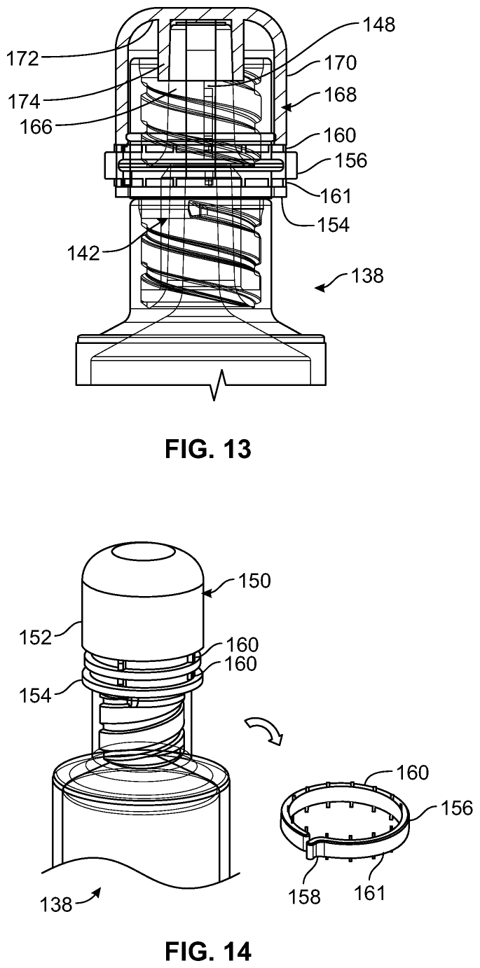

As shown in FIG. 12, in the end cap assembly embodiment 150, an inner surface 162 of the axial wall of the end cap embodiment 152 defines a male sprue 164. When the end cap embodiment 152 covers the male, second Luer connector 146, the male sprue 164 seals the second lumen 148 within the male Luer tip 166 to prevent fluid leakage and maintain sterility of the covered Luer connector. An alternative end cap assembly 168 embodiment, shown in FIG. 13, also seals the male Luer tip 166. In this alternative embodiment, the end cap 170 has an inner axial wall surface 172 that defines a circumferential collar 174, which is in sealing engagement with a corresponding outer circumferential surface of the male Luer tip 166.

In other embodiments, the tamper evident end cap structure comprises shrink-wrap plastic film covering the end cap, or a series of perforations, or thinner wall construction, or another type of frangible junction joining the end cap and the snap-on band. In some embodiments, the snap-on band is integrally formed within the housing 142. In some embodiments, the snap-on band 154 has a contrasting visual color (e.g., red) to remind clinicians that either end cap 152 or 170 embodiment has been removed from the syringe coupler 140 and that its corresponding male, second Luer connector 146 may not be sterile.

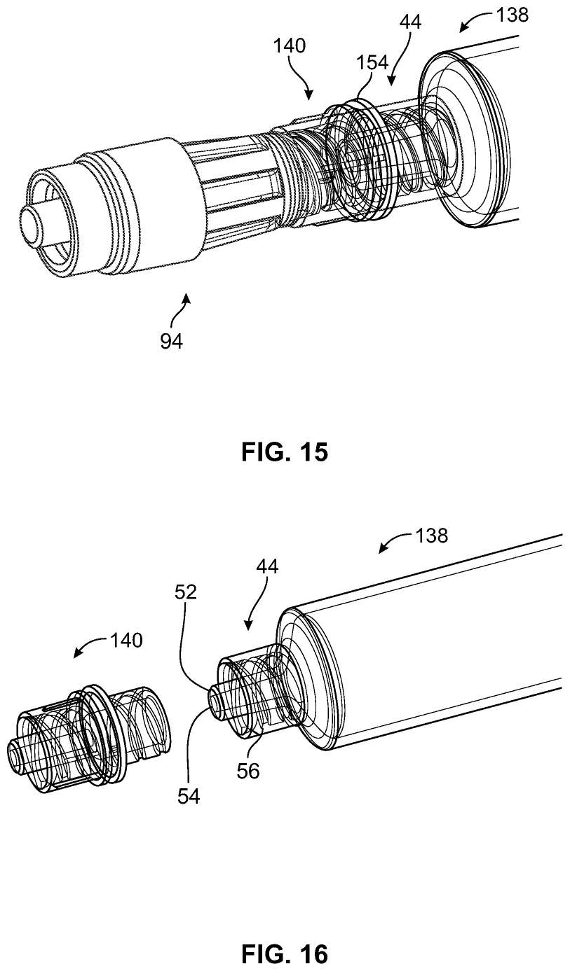

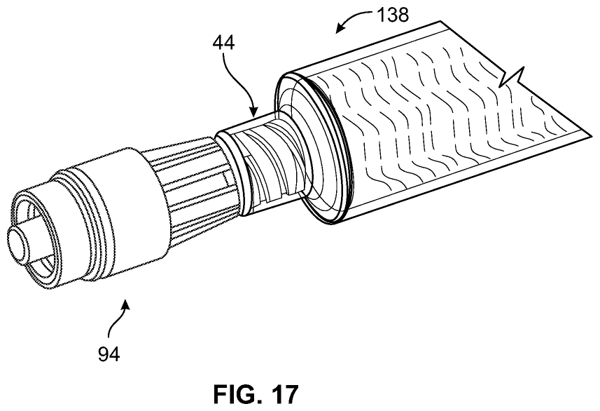

Use of the flush syringe 138 embodiment is as follows, referring to FIGS. 11, and 14-17 in sequential order. For brevity, SASH hub scrubbing procedures followed during the flush syringe 138 use are not described. The clinician prepares the flush syringe 138 by peeling the finger tab 158 of the tear-off ring 156 away from the syringe coupler 140; this tears the respective shear-off connectors 160 and 161 (FIG. 11). The tear-off ring 156 is separated from the snap-on band 154 and the end cap 152 (FIG. 14). The syringe coupler 140 is coupled to the patient port 94 enabling a first flushing of the port (FIG. 15). Upon completion of the first flushing procedure, the syringe coupler 140 is separated from the patient port and the barrel Luer connector 44 and thereafter it is discarded (FIG. 16). After administration of medication to the patient, the barrel Luer connector 44 is directly coupled to the patient port 94, which enables administration of the second flushing procedure (FIG. 17), as was described with respect to the flush syringe 30 embodiment of FIGS. 1-6.

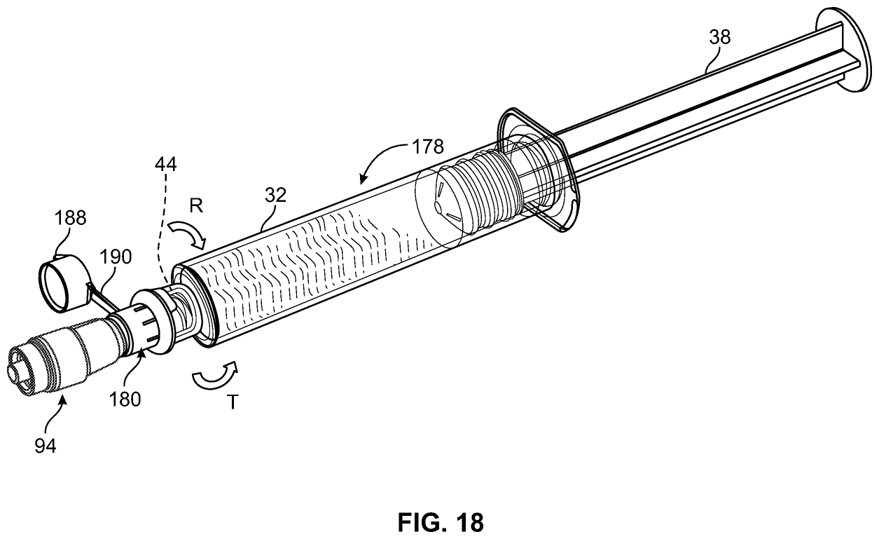

FIGS. 18-22 depict another embodiment of a flush syringe 178, with a twist-lock mechanism for preventing inadvertent axial separation of the barrel Luer connector 44 from the syringe coupler 180 when the twist-lock mechanism is engaged in a first position, prior to threading engagement of the syringe coupler into connection with a patient port 94. Conversely, when the syringe coupler 180 is unthreaded/detached from the patient port 94, the twist-lock mechanism passively disengages in a second position, which facilitates passive separation of the syringe coupler 180 from the barrel Luer connector 44. By passively separating the syringe coupler 180 from the barrel Luer connector 44 after its removal from a patient port or other VAD, it cannot be reused inadvertently after its initial use of its first Luer connector.

As shown in FIG. 18, flush syringe 178 is affixed to the patient port 94 by relative clockwise rotation (double arrow T) and engages the twist-lock mechanism in its first position. When flush syringe 178 is removed from patient port 94 by relative counterclockwise rotation (double arrow R) it translates the twist-lock mechanism to its second position.

The syringe barrel 32, syringe plunger 37 and the barrel Luer connector 44 of the flush syringe 178 are identical to those of the previously described flush syringe embodiments 30 and 103 of FIGS. 1-8. The syringe coupler 180 comprises a housing 182, with a female, first Luer connector 184 on a proximal end of the housing that is coupled to the barrel Luer connector 44. The housing 182 further comprises a male, second Luer connector 186 for coupling to a patient port 94, a flip cap-type end or storage cap 188 and a flip cap hinge 190. The structure, function and operation of the flip cap 188 and the flip cap hinge 190 are the same as the flip cap 80 and flip cap hinge 92 of the flush syringe 30 of FIGS. 1-6. In various embodiments, internal structure and fluid passage through the syringe coupler 180 from the internal syringe cavity 40 into the patient port 94 is identical to those of the syringe coupler embodiments 46 and 104 of FIGS. 1-8.

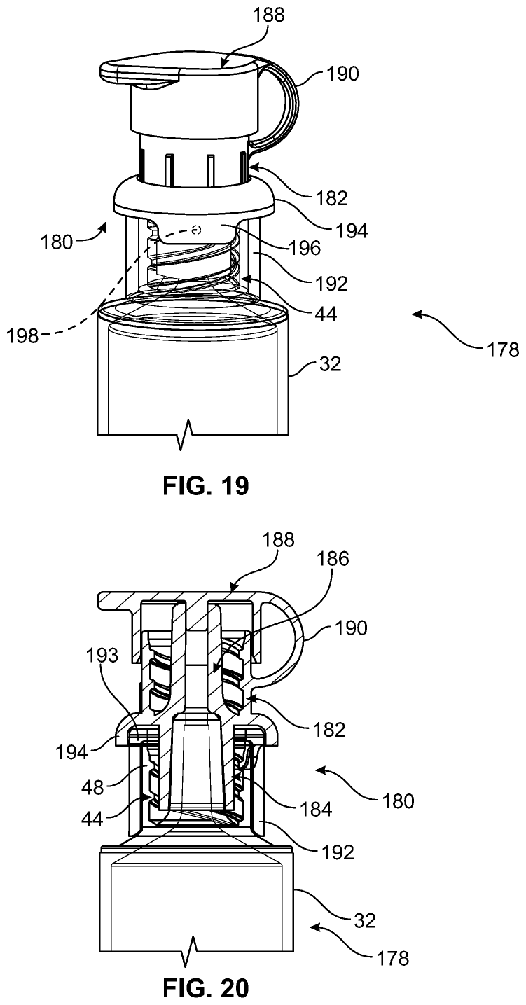

Referring more specifically to FIGS. 19-22, the twist-lock mechanism comprises a hollow coupler sleeve 192, having a proximal end with an inner surface rigidly therein an exterior circumferential surface of the collar 48 of the male, barrel Luer connector 44. In this embodiment, coupler sleeve 192 is a separate component rigidly coupled to the barrel Luer connector 44. In this way, coupler sleeve 192 allows incorporation of the twist-lock mechanism in existing flush syringes. In other embodiments, the coupler sleeve 192 is integrally formed within the barrel Luer connector 44. The twist-lock mechanism further comprises a cup-like housing skirt 194 that is formed in the housing 182, intermediate the respective first, female 184 and second, male 186 Luer connectors. The housing skirt 194 circumscribes the housing 182, about the central axis, and the first, female Luer connector 184, and extends axially toward the proximal end of the housing. Housing skirt 194 has an axially extension 196, which retains a skirt pin 198 that extends radially inwardly toward the central axis of housing 182. A distal end 193 and outer surface of the coupler sleeve 192 is received and rotatable within the housing skirt 194.

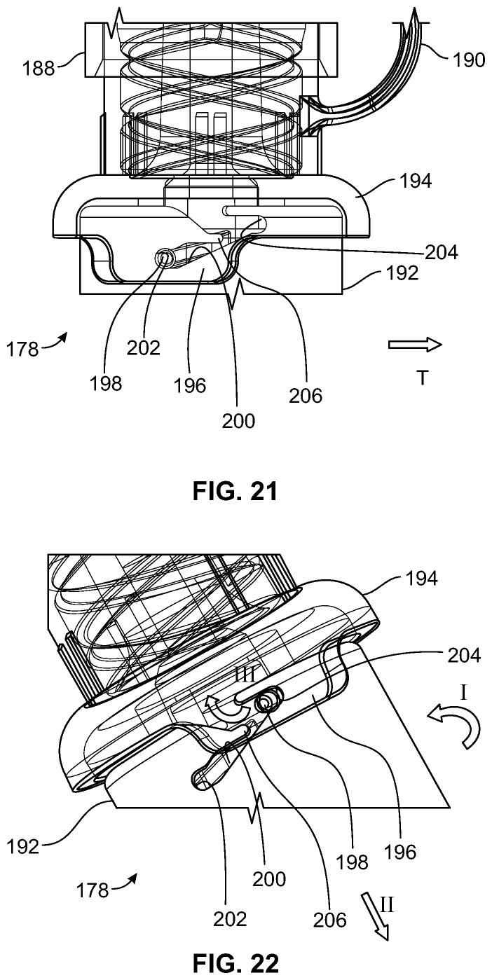

As the syringe barrel 32 is rotated relative to the housing 182 of the syringe coupler 180, such as during connection or disconnection of the barrel Luer connector 44 to the female, first Luer connector 184, the coupler sleeve 192 that is rigidly coupled to the barrel Luer connector also rotates relative to the housing skirt. When the respective opposing threads of the barrel Luer connector 44 and the female, first Luer connector 184 are not mated, the collar is separable axially from the housing 182 and the housing skirt 194. Referring to FIGS. 21 and 22, axial separation and relative rotation between the coupler sleeve 192 and the housing skirt 194 is restrained by motion of the skirt pin 198 that is received in and rides in a generally S-shaped camming slot 200 formed in an outer circumferential surface of the coupler sleeve 192. Other flush syringe embodiments incorporate plural pairs of skirt pins 198 and camming slots 200.

In FIG. 21, the skirt pin 198 is oriented in the previously described the first position 202 within the camming slot 200 when the first, female Luer connector 184 and the barrel Luer connector 44 are fully tightened in fluid-sealed engagement (double arrow T). The skirt pin 198 orientation within the first position 202, (as well as the tightened first, female Luer connector 184 and the barrel Luer connector 44), prevents relative axial separation of the housing 182 and the coupler sleeve 192/syringe. As shown in FIGS. 18 and 22, when the syringe coupler 180 is unscrewed from the patient port 94 (double arrow R), the coupler sleeve 192 also rotates in the same direction (double arrow I), causing the skirt pin 198 to ride along the camming slot 200 to its second position 204, after biasing the pawl 206 towards the housing skirt 194. Pawl 206 prevents inadvertent movement of the skirt pin 198 from the first position 202 into the second position 204. When the syringe coupler 180 is fully unscrewed from both the patient port 94 and the syringe barrel 32, retraction of the flush syringe 30 in the axial direction (double arrow II) passively separates the syringe coupler 180 from the patient port 94 but does not separate the syringe coupler from the syringe barrel. This is because skirt pin 198 retention within the second position 204 prevents total axial separation of the syringe coupler 180 and the flush syringe 30. When it is desired to separate the syringe coupler 180 from the syringe barrel 32, the skirt pin 198 is translated out of the camming slot 200, along the path designated by the double arrow III. The separated flush syringe 30 is now available for direct connection to patient port 94, for administration of a second port flushing procedure, as was described with respect to the flush syringe 30 embodiment of FIGS. 1-6.

Components of the flush syringe embodiments 30, 103, 138, and 178, and the other embodiments described herein, are constructed by injection molding or vacuum thermal forming. Exemplary materials used to construct the syringe couplers and their end/storage caps include thermoplastic polymers. Hollow sleeve 116 is an elastomeric sleeve that is constructed by compression molding of cross-linked rubber or thermoplastic elastomer materials. Generally, the components of the flush syringe embodiments 30, 103, 138, and 178 are constructed with types of medical grade, plastic materials such as polycarbonate, polypropylene, polyethylene, glycol-modified polyethylene terephthalate, acrylonitrile butadiene styrene or any other moldable plastic material used in medical devices.

One or more of the flush syringe embodiments described in this disclosure are manufactured as follows. In one exemplary manufacturing embodiment, referring to the flush syringe 30 of FIGS. 1-6, a flush syringe 30 is provided, generally comprising a syringe barrel 32 with a male, barrel Luer connector 44 oriented on a distal end 34 of the syringe barrel, an open, proximal end 36, and a syringe cavity 40 within the barrel. In some embodiments, the barrel Luer connector 44 has a male, barrel Luer tip 50 defining an outlet lumen 54 in communication with the syringe cavity 40. In some embodiments, an existing, off-the-shelf, 10 ml capacity flush syringe is utilized for manufacture of the sequential delivery flush syringe 30 of the present disclosure.

The manufactured flush syringe 30 embodiment incorporates a syringe coupler 46, which includes the previously described a housing 58 having proximal 60 and distal 62 axial ends and a central axis; a female, first Luer connector 64 on the proximal end of the housing, oriented coaxial with the central axis, having a first lumen 68 defined therein. The syringe coupler 46 further comprises a male, second Luer connector 72 on the distal end of the housing, oriented coaxial with the central axis, with a male Luer tip 74 thereof having a second lumen 75. The first 68 and second 75 lumens are in fluid communication with each other. The female, first Luer connector 64 is coupled to the barrel Luer connector 44. A storage cap or end cap (here flip cap 80) is coupled to the male, second Luer connector 72 of the syringe coupler 46. The syringe cavity 40 is pre-filled with a flushing solution 42 prior to insertion of the syringe plunger 38 into the syringe barrel 32 within the syringe cavity. In some embodiments, the completed, pre-filled flush syringe is sterilized and packaged in sterile packaging 31. In some embodiments, plural flush syringes 30 are packaged in a shared, sterile packing container.

In another exemplary manufacturing method, referring to the flush syringe 138 of FIGS. 9-17, the monolithic, molded, tamper-evident, end cap assembly 150 is coupled to its syringe coupler 140 by advancing the snap-on band 154 over the housing 142. Other steps for manufacturing the flush syringe 138 are the same as those for manufacturing the flush syringe 30.

In another exemplary manufacturing method, referring to the flush syringe 178 of FIGS. 18-22, the coupler sleeve 192 is rigidly coupled to the male, barrel Luer connector 44 prior to coupling the syringe coupler 180 to the barrel Luer connector 44. As the barrel Luer connector 44 is advanced into axial alignment with and threaded into the female, first Luer connector 184, the axial extension 196 of the housing skirt 194 is biased outwardly away from the syringe coupler 180, clearing the skirt pin 198 from contact with the coupler sleeve 192. When skirt pin 198 is radially aligned with the camming slot 200, the axial extension 196 is allowed to relax to its unbiased state, thereby orienting the skirt pin within the camming slot 200. The syringe barrel 32 is rotated counterclockwise to orient the skirt pin 198 into the first position 202 within the camming slot 200. Other steps for manufacturing the flush syringe 138 are the same as those for manufacturing the flush syringe 30.

Reference throughout this specification to “one embodiment,” “certain embodiments,” “various embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in various embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are to be interpreted broadly; they encompass direct and indirect mountings, connections, supports, and couplings. Further, “connected” and “coupled” are not restricted to physical, mechanical, or electrical connections or couplings.

Although the disclosure herein provided a description with reference to embodiments, it is to be understood that these embodiments are merely illustrative of the principles and applications of the disclosure. It will be apparent to those skilled in the art that various modifications and variations can be made to the present disclosure without departing from the spirit and scope thereof. For example, the structural features and operation of specific syringe coupler embodiments 46, 104, 140, and 180 have been described herein. Various male and female, threaded Luer connectors that have been described are integrated into the syringe barrels 32 as well as the syringe couplers 46, 104, 140, and 180. In some embodiments those Luer connectors are not threaded Luer connectors. Similarly, while the tamper-evident, end cap assembly 150 has been described as comprising a tear-of ring 156, other embodiments utilize other types of tamper-evident fittings. Other flush embodiments incorporate both the tamper-evident end cap structures and the passive separation of the syringe coupler from the syringe barrel structures disclosed herein. It is intended that the present disclosure include modifications and variations that are within the scope of the appended claims and their equivalents. The appended claims are not limited to the exemplary embodiment details of construction and the arrangement of components set forth in the description or illustrated in the drawings.

What is claimed is:

1. A flush syringe for intravenous fluid administration to a patient, comprising:
a syringe barrel with a distal end, an open proximal end, and a syringe cavity within the syringe barrel;
a syringe plunger translatable within the syringe cavity;
a male, barrel Luer connector oriented on the distal end of the syringe barrel, having a male, barrel Luer tip defining an outlet lumen in communication with the syringe cavity;
a syringe coupler, including:
a housing having proximal and distal axial ends, and a central axis;
a female, first Luer connector on the proximal axial end of the housing, coaxial with the central axis, having a first lumen defined therein;
a male, second Luer connector on the distal end of the housing, coaxial with the central axis, a male Luer tip thereof having a second lumen defined therein that is in fluid communication with the first lumen;
a storage cap, coupled to the housing, covering the male, second Luer connector and sealing the second lumen;
the female, first Luer connector of the housing coupled to the male, barrel Luer connector, for delivery of a flushing solution retained within the syringe cavity through the outlet lumen of the male, barrel Luer connector, into the first and second lumens of the housing;
the male, second Luer connector of the housing adapted for coupled insertion into a corresponding needle-free IV connector of a patient for delivery through the second lumen of at least a portion of the flushing solution retained within the syringe cavity into the IV connector; and
the syringe coupler selectively removeable from the male, barrel Luer connector, thereby allowing direct coupling of the latter to the corresponding needle-free IV connector for delivery of additional flushing solution retained within the syringe cavity into the IV connector.

2. The flush syringe of claim 1, further comprising a hollow sleeve retained within the first lumen; a proximal axial end of the hollow sleeve projecting out of the first lumen towards the proximal axial end of the housing and inserted within the outlet of the male, barrel Luer tip.

3. The flush syringe of claim 1, the storage cap further comprising a selectively removeable flip cap coupled to the distal end of the syringe coupler.

4. The flush syringe of claim 3, further comprising a cap hinge coupled to the flip cap and the coupler housing, for tethering the flip cap to the coupler housing.

5. The flush syringe of claim 1, further comprising a circumferential cup projecting axially away from the distal end of the housing, defining a distal axial surface; and a peel lid-type storage cap coupled to the distal axial surface of the circumferential cup and the second lumen.

6. The flush syringe of claim 1, the storage cap further comprising a tamper evident, selectively removeable end cap assembly.

7. The flush syringe of claim 6, the tamper evident, end cap assembly further comprising: an end cap; a frangible, tear-off ring connected to the end cap by at least one first, shear-off connector and connected by second, at least one shear-off connector to the syringe coupler.

8. The flush syringe of claim 7, the end cap assembly further comprising:
a monolithic structure, end cap assembly with a snap-on band circumscribing the syringe coupler; a split, tear-off ring incorporating a finger tab; a plurality of integrally formed, first, shear-off connectors joining the end cap and the split, tear-off ring; and a plurality of integrally formed, second, shear-off connectors joining the split, tear-off ring and the snap-on band.

9. The flush syringe of claim 1, further comprising a twist-lock mechanism coupling the proximal end of housing to the barrel Luer connector, which when engaged in a first position, prevents their axial separation and disengages in a second position when removing the female, first Luer connector of the housing from the male, barrel Luer connector, which allows such axial separation.

10. The flush syringe of claim 9, the twist-lock mechanism further comprising:
the housing defining a housing skirt intermediate the respective first, female and second, male Luer connectors, the housing skirt circumscribing the housing and the first, female Luer connector about the central axis and extending axially toward the proximal end of the housing;
a skirt pin extending radially inwardly toward the central axis;
a hollow coupler sleeve having a proximal end with an inner surface thereof receiving rigidly therein an exterior circumferential surface of the male, barrel Luer connector, and a distal end with an outer surface thereof received and rotatable within the housing skirt;
a camming slot formed in the outer surface of the distal end of the coupler sleeve, receiving and capturing the skirt pin therein, with the skirt pin oriented in the first position within the camming slot when the first, female Luer connector and the barrel Luer connector are fully tightened in fluid-sealed engagement, and with the skirt pin oriented in the second position in the camming slot when the first, female Luer connector and the barrel Luer connector are disconnected from each other.

11. The flush syringe of claim 10, further comprising:
threaded barrel, first, female and second, male Luer connectors;
the male, barrel Luer connector and the male, second Luer connector respectively having threaded collars circumscribing respective male Luer tips thereof;
the hollow coupler sleeve receiving rigidly therein an exterior circumferential surface of the outer threaded collar of the corresponding male, barrel Luer connector, or formed integrally with the male, Barrel Luer connector;
the skirt pin oriented in the first position within the camming slot when the first, female Luer connector and the barrel Luer connector are fully screwed together and the skirt pin oriented in the second position when first, female Luer connector and the barrel Luer connector are fully screwed from each other; and
a pawl oriented within the camming slot intermediate the first and second positions, for inhibiting inadvertent movement of the pin to the second position.

12. A method for manufacturing a flush syringe, comprising:
providing a syringe barrel with a male, barrel Luer connector oriented on a distal end of the syringe barrel, an open proximal end, and a syringe cavity within the barrel, the barrel Luer connector having a male, barrel Luer tip defining an outlet lumen in communication with the syringe cavity;
providing a syringe coupler, including:
a housing having proximal and distal axial ends and a central axis;
a female, first Luer connector on the proximal end of the housing, coaxial with the central axis, having a first lumen defined therein;
a male, second Luer connector on the distal end of the housing, coaxial with the central axis, a male Luer tip thereof having a second lumen defined therein that is in fluid communication with the first lumen;
coupling the female, first Luer connector to the barrel Luer connector;
coupling a selectively removeable storage cap to the housing, covering the male, second Luer connector of the syringe coupler and sealing the second lumen;
pre-filling the syringe cavity with a flushing solution;
inserting a translatable syringe plunger into the syringe barrel within the syringe cavity.

13. The method of for manufacturing the flush syringe of claim 12, further comprising sterilizing the pre-filled flush syringe after insertion of the syringe plunger; and packaging the now sterilized, pre-filled flush syringe in a sterile packaging container.

14. A method flushing a patient port during intravenous fluid administration to a patient by using the flush syringe of claim 1, comprising:
disinfecting a hub of a patient port having a hub Luer connector;
removing the storage cap from the syringe coupler;
coupling the male, second Luer connector of the syringe coupler to the hub Luer connector of the patient port;
dispensing a first portion of pre-filled flushing solution retained within the syringe cavity through the male, second Luer connector into the patient port by depressing the syringe plunger;
de-coupling the male, second Luer connector from the hub Luer connector;
disinfecting the hub of the patient port;
administering one or more drugs through the hub with drug dosing syringes, disinfecting the hub after delivery of each drug;
decoupling the female, first Luer connector of the syringe coupler from the male, barrel Luer connector of the syringe, thereafter, discarding the syringe coupler;
disinfecting the hub of the patient port;
coupling the male, barrel Luer connector to the hub Luer connector of the patient port and dispensing a second portion of the pre-filled flushing solution retained with the syringe cavity through the male, barrel Luer connector into the patient port by depressing the syringe plunger;
decoupling the male, barrel Luer connector from the hub Luer connector of the patient port, thereafter, discarding the syringe;
disinfecting the hub of the patient port; and
capping the hub of the patient port with a sterilized hub cap.

15. The method for flushing a patient port of claim 13, further comprising: removing the flush syringe from sterile packaging and decoupling the storage cap from the male, second Luer connector of the syringe coupler, prior to coupling latter to the hub Luer connector.

16. A syringe coupler for intravenous fluid administration to a patient, comprising:
a syringe coupler, including:
a housing having proximal and distal axial ends, and a central axis;
a female, first Luer connector on the proximal end of the housing, coaxial with the central axis, having a first lumen defined therein;
a male, second Luer connector on the distal axial end of the housing, coaxial with the central axis, a male Luer tip thereof having a second lumen defined therein that is in fluid communication with the first lumen;
a storage cap, coupled to the housing, covering the male, second Luer connector and sealing the second lumen;
the female, first Luer connector of the housing adapted for coupled receipt of a male Luer tip of a Luer connector of a flush syringe, whereby an outlet lumen formed within the syringe's male Luer tip is in fluid communication with the first and second lumens of the housing, for delivery of flushing solution from the flush syringe through the second lumen; and
the male, second Luer connector of the housing adapted for coupled insertion into a corresponding needle-free IV connector of a patient, for delivery of flushing solution therefrom into the IV connector, after removal of the storage cap.

17. The syringe coupler of claim 16, the storage cap further comprising a selectively removeable flip cap coupled to the distal end of the syringe coupler.

18. The syringe coupler of claim 16, further comprising a circumferential cup projecting axially away from the distal end of the housing, defining a distal axial surface; and a peel lid-type storage cap coupled to the distal axial surface of the circumferential cup and the second lumen.

19. The syringe coupler of claim 16, the storage cap further comprising a tamper evident, selectively removeable end cap assembly.

20. The syringe coupler of claim 19, the end cap assembly further comprising: an end cap; a frangible, tear-off ring connected to the end cap by at least one first, shear-off connector and connected by second, at least one shear-off connector to the syringe coupler.

21. The syringe coupler of claim 20, the end cap assembly further comprising:
a monolithic, snap-on band circumscribing the syringe coupler;
a split, tear-off ring incorporating a finger tab;
a plurality of integrally formed, first, shear-off connectors joining the end cap and the split, tear-off ring; and
a plurality of integrally formed, second, shear-off connectors joining the split, tear-off ring and the snap-on band.

22. The syringe coupler of claim 16, further comprising a twist-lock mechanism adapted for coupling the proximal end of housing to a barrel Luer connector of a flush syringe, which when engaged in a first position, prevents their axial separation and disengages in a second position when removing the female, first Luer connector of the housing from the male, barrel Luer connector, which allows such axial separation.

23. The syringe coupler of claim 22, the twist-lock mechanism further comprising:
the housing defining a housing skirt intermediate the respective first, female and second, male Luer connectors, the housing skirt circumscribing the housing and the first, female Luer connector about the central axis and extending axially toward the proximal end of the housing;
a skirt pin extending radially inwardly toward the central axis;
a hollow coupler sleeve having a proximal end with an inner surface thereof adapted for receiving rigidly therein an exterior circumferential surface of a male, barrel Luer connector, and a distal end with an outer surface thereof received and rotatable within the housing skirt;
a camming slot formed in the outer surface of the distal end of the coupler sleeve, receiving and capturing the skirt pin therein, with the skirt pin oriented in the first position within the camming slot when the first, female Luer connector and the barrel Luer connector are fully tightened in fluid-sealed engagement, and with the skirt pin oriented in of the second position within the camming slot when the first, female Luer connector and the barrel Luer connector are disconnected from each other.

24. The syringe coupler of claim 23, further comprising a pawl oriented within the camming slot intermediate the first and second positions, for inhibiting inadvertent movement of the pin to the second position.
